SEQUENCE LISTING (3 sequences)

SEQ ID NO: 1 - protein, 387 aa, Artificial [sequence omitted]

SEQ ID NO: 2 - protein, 508 aa, Artificial
  MISC_FEATURE (1)..(20): preceding peptide with 6x His tag
MGSSHHHHHHSSGLVPRGSHMGSEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPMGVVRPLTEVEMDHYREPFLKPVDREPLWRFPNELPIAGEPANIVALVEAYMNWLHQSPVPKLLFWGTPGVLIPPAEAARLAESLPNCKTVDIGPGLFLLQEDNPDLIGSEIARWLPGLAGLEVLFQGPLEMDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 3 - protein, 182 aa, Artificial
  MISC_FEATURE (1)..(182): The sequence is based on an AGT mutant described in Gronemeyer et al., Protein Eng. Des. Sel. 19, 309-318 (2006), containing the following additional mutations Y114R; K131S; S135D; V148E; E159M.
MDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISRSHLAALAGNPAATAAVSTALDGNPVPILIPCHREVQGDLDVGGYMGGLAVKEWLLAHEGHRLGKPGLG

(19) United States
(12) Patent Application Publication
(10) Pub. No.: US 2010/0075394 A1
(43) Pub. Date: Mar. 25, 2010

(54) Method of Crosslinking Two Objects of Interest

(75) Inventors: Kai Johnsson, Lausanne (CH); Christopher Chidley, Crissier (CH); Katarzyna Mosiewicz, Chavannes-Pres-Renens (CH)

Correspondence Address: HARRIET M. STRIMPEL, D. Phil., New England Biolabs, Inc., 240 COUNTY ROAD, IPSWICH, MA, 01938-2723, US

(73) Assignee: ECOLE POLYTECHNIQUE FEDERALE DE LAUSANNE, Lausanne (CH)

(21) Appl. No.: 12/594,256

(22) PCT Filed: Apr. 4, 2008
(86) PCT No.: PCT/EP2008/054088

§ 371 Date: Oct. 1, 2009

(30) Foreign Application Priority Data
Apr. 4, 2007 (EP) 07105672.5

(51) Int. Cl. C12N 9/10; C12N 1/21; C12N 1/19; C07K 14/00; C07K 19/00; C07K 16/46

(52) U.S. Cl. 435/193; 530/350; 435/252.33; 435/254.21; 530/395; 530/402; 530/391.1

(57) ABSTRACT

The invention provides a method of crosslinking two objects of interest, comprising the steps of: i) providing a fusion protein comprising at least a first protein and a second protein, wherein both the first and the second protein are, based on their structure and function, capable of forming a covalent bond with given substrates, and which first and second proteins are of substantially non-overlapping substrate selectivity, preferably of different substrate specificity; ii) providing a first object of interest, comprising a substrate moiety for the first protein of the said fusion protein, and providing a second object of interest, comprising a substrate moiety for the second protein of the said fusion protein; and iii) reacting said first protein of the fusion protein with the substrate moiety of said first object, and reacting said second protein of the fusion protein with the substrate moiety of said second object, thereby covalently crosslinking the first object to the second object via the said fusion protein. Most prominent applications of the disclosed method are, due to the straight-forward, reliable, directional and fast crosslinking reactions: the derivatization of cells, antibodies and the crosslinking of proteins.

### FIELD OF INVENTION

In broad sense, the present invention relates to methods of covalently crosslinking two objects of interest in a reliable, selective and directional manner, mediated by a protein, especially a fusion protein. In particular, the invention concerns methods for use in derivatization of biological molecules, e.g. proteins, antibodies, viruses or cells, to methods of crosslinking of object proteins, methods to directionally couple physical or chemical objects for applications in nanotechnology and to methods of binding of a molecular object of interest to a solid support.

### BACKGROUND OF THE INVENTION

The specific connection or crosslinking of two or multiple objects which may be (i) biomolecules such as proteins and DNA, (ii) cells and viruses, as well as (iii) nanomaterials and synthetic molecules, is an omnipresent issue in biotechnology and nanotechnology. One way to achieve the connection of objects is through the mediation of proteins. In this case the protein must possess an affinity for the two objects. This affinity can be the natural affinity of the protein towards the object, such as the affinity of antibodies towards their antigens. As the antibody has two binding sites, it can connect two antigens in a non-covalent manner. Alternatively, the objects might be derivatized with ligands that are specifically recognized by some protein. The best know example for such a protein-ligand pair is streptavidin. Two different objects can be biotinylated and then connected through the tetrameric streptavidin or avidin, which possesses four different binding sites. Such biotinylated objects can be cells, proteins, physical objects or synthetic molecules. The biotinylation can be achieved through a simple coupling of an activated biotin derivative, such as a commercially activated ester, or through the action of a protein. The addition of streptavidin to such biotinylated objects then leads to the connection mediated by the tetrameric streptavidin. This and related approaches suffer from two main points: Firstly, the connection is not covalent and irreversible and thus is not stable under conditions in which the protein that mediates the connection is denatured. Such conditions can be high temperature or organic solvents. Secondly, the connection/crosslinking is not directional when multiple objects are present. For example, addition of streptavidin to two different biotinylated proteins A and B will lead to the formation of a number of different complexes (i.e. crosslinking of A with A, A with B, B with A, and higher aggregates), their relative frequency determined by the relative concentrations of the biotinylated proteins and streptavidin.

An example for the importance of connecting different objects is antibody-based bioassays. Antibodies (the first object) are the key element in numerous bioassays and bind non-covalently to a specific object of interest. In general, they need to be derivatized with a probe (the second object) that allows for detection of the antibody. Such probes can be proteins, fluorophores, gold particles etc. The probe is attached to the antibody either through direct chemical coupling, or the probe is coupled to a secondary antibody that specifically recognizes and binds to the primary antibody. In any case, chemical derivatization of an antibody with a probe is a prerequisite for this technique, and for each different experiment a differently labelled antibody needs to be prepared or purchased.

On the other hand, the transfer of a label from substrates to fusion proteins consisting of an O⁶-alkylguanine-DNA alkyltransferase (hereinafter: AGT) and a protein of interest is known inter alia from WO 02/083937 (PCT/GB02/01636), WO 2004/031404 (PCT/EP03/10859), WO 2004/031405 (PCT/EP03/10889) and WO 2005/085431 (PCT/EP2005/050889), respectively; the disclosure of these documents is incorporated by reference into the present application. In these documents, AGT is fused to a protein of interest, and the AGT is used to covalently attach a label to the fusion protein which subsequently allows for detection and/or manipulation (e.g. purification or immobilization) of the fusion protein. Most recently, the fusion protein ^{M}AGT-DEVD-^{L}AGT has been labelled with two different fluorophores and an intramolecular FRET has been detected (Heinis et al., ACS Chemical Biology 1(9), 2006, 575-84).

Recently, mutants of AGT (hereinafter ACTs) were developed (Patent application number EP06117779, entitled “Labelling of fusion proteins with synthetic probes”, incorporated herein by reference) that specifically react with benzylcytosine (hereinafter BC) derivatives and which allow the labelling of ACT fusion proteins using BC derivatives the same way as AGT fusion proteins can be labelled with benzylguanine (hereinafter BG) derivatives. Importantly, the reactivity of ACT versus BG derivatives is below 1% of the reactivity versus BC derivatives and the reactivity of AGT versus BC derivatives is below 1% the reactivity of so AGT versus BG. ACT and AGT thus have substantially non-overlapping substrate specificity.

Yet another approach of covalently attaching a label to a protein is the HaloTag™ (Promega Corporation, 2800 Woods Hollow Road, Madison, Wis., USA), described in Los et al., Cell Notes 11, 2005, 2-6, WO 2006/093529 and WO 2004/072232. The system is based on a genetically engineered hydrolase, in which the final hydrolysis step is impaired and the label thus remains covalently attached to the hydrolase.

Concluding, the HaloTag™, the AGT system and the ACT system are known in the art for covalently attaching a label (i.e. a tag which provides a possibility of detection or further manipulation, but which label is not of interest by itself) to a fusion protein comprising a protein of interest and the HaloTag™ or AGT, respectively.

Concluding, no method of covalently crosslinking two objects of interest in a reliable, selective and directional manner is currently available.

It is thus an object of the invention to overcome the above-mentioned drawbacks of prior art methods of crosslinking/connecting two objects of interest, i.e. to provide a method which works reliably, selectively and directionally, and especially to provide a method of crosslinking two objects of interest for use in derivatization of e.g. antibodies, viruses or cells, methods of crosslinking of physical and chemical objects on the nanometer scale and methods of binding of an object of interest to a solid support or a cell.

### SUMMARY OF THE INVENTION

This invention provides a fusion protein comprising at least a first protein and a second protein, wherein both the first and the second protein are, based on their structure and function, capable of forming a covalent bond with given substrates, and which first and second proteins are of substantially non-overlapping substrate selectivity, preferably of different substrate specificity; with the provisio that the fusion protein is not ^{M}AGT-DEVD-^{L}AGT or ^{L}AGT-DEVD-^{M}AGT (^{M}AGT-DEVD-^{L}AGT and ^{L}AGT-DEVD-^{M}AGT do not contain two proteins with different selectivity/specificity; thus, crosslinking would have to be carried out sequentially and could not be reliably achieved in mixtures of both substrates for ^{M}AGT and ^{L}AGT, respectively.)

As noted above, such fusion proteins are not known in the art, to the best of applicant's knowledge. Neither the AGT and ACT nor the HaloTag™ have been reported in fusion proteins to allow for connecting two objects of interest to each other. As will be outlined below in any detail, these fusion proteins serve as reliable, selective and directional tools for dual, covalent crosslinking with different objects of interest (especially proteins), also allowing inter alia for novel approaches in derivatization of cells, viruses, antibodies and any object (biological or synthetic) that can be derivatized with the substrate reacting with the protein.

The invention moreover provides a recombinant DNA sequence encoding for the said fusion protein; an expression vector containing an expression cassette encoding for the said fusion protein; and a prokaryotic or eukaryotic host cell line, enabled to functionally express the said fusion protein and/or transformed with the said expression vector.

Yet another aspect of the invention concerns a kit-of-parts, comprising, besides the said fusion protein and/or the said expression vector and/or the said host cell line, at least one molecule comprising a substrate moiety for at least either the first or the second protein of the said fusion protein. This substrate can be a biomolecule, a cell, a virus or any other synthetic or natural object derivatized with the substrate to allow for the covalent and specific crosslinking/connecting of two objects.

As will be outlined below in any detail, the molecule comprising a substrate moiety is used for modifying the respective object of interest suchlike that this object of interest then presents a substrate moiety for the said fusion protein, thereby forming a covalent crosslink between the object of interest and the fusion protein. Preferably, the kit-of-parts encompasses at least two such molecules, the one molecule comprising a substrate moiety for the first protein of the said fusion protein, and the other molecule comprising a substrate moiety for the second protein of the said fusion protein.

Yet a further aspect of the present invention thus concerns a method of crosslinking two objects of interest, comprising the steps of:
i) providing a fusion protein comprising at least a first protein and a second protein, wherein both the first and the second proteins are, based on their structure and function, capable of forming a covalent bond with given substrates, and which first and second proteins are of different substrate selectivity, preferably of different substrate specificity;
ii) providing a first object of interest, comprising a substrate moiety for the first protein of the said fusion protein, and providing a second object of interest, comprising a substrate moiety for the second protein of the said fusion protein;
iii) reacting said first protein of the fusion protein with the substrate moiety of said first object, and reacting said second protein of the fusion protein with the substrate moiety of said second object, thereby covalently crosslinking the first object to the second object via the said fusion protein.
   Both covalent bonds can be formed simultaneously due to different substrate selectivity or specificity, i.e. no stepwise labelling is necessary. However, the crosslinking can also be achieved through sequential reactions; here, the order of the reactions is not relevant.

The present invention moreover provides a method of modifying a first object and/or a second object, for use with a fusion protein comprising at least a first protein and a second protein, wherein both the first and the second protein are, based on their mechanism of enzymatic catalysis, capable of forming a covalent bond with given substrates, and which first and second proteins are of different substrate selectivity, preferably of different substrate specificity, comprising the steps of:
i) transferring to the first object a substrate moiety for the first protein of the said fusion protein; and/or
ii) transferring to the second object a substrate moiety for the second protein of the said fusion protein.

### DESCRIPTION OF THE FIGURES

SEQ ID NO:1 ^{M}AGT-DEVD-^{L}AGT

SEQ ID NO:2 HaloTag™-AGT (covalin)

SEQ ID NO:3 ACT1

FIG. 1. An embodiment of the present invention. Two objects (A) and (B) are chosen in step (I.), such as e.g. cells, antibodies, solid surfaces, proteins, proteins, etc. In step (II.), these objects are modified with suitable substrate moieties, as will be outlined below in more detail. In step (III.), object (A) is modified in the present example with an O⁶-benzylguanine derivative which is a substrate for AGT. R² represents hydrogen, β-D-2′-deoxyribosyl or β-D-2′-deoxyribosyl which forms part of a deoxyribonucleotide, preferably having a length between 2 and 99 nucleotides; R¹ represents a linker group as commonly applied in the art, for example a flexible linker group such as a substituted or unsubstituted alkyl chain or a polyethylene glycol. Object (B) is, in the present example, modified with an aliphatic halogenated (here: chlorinated) alkyl chain; the modified object (B) is thus a substrate for the modified hydrolase of the HaloTag™. In step (IV.), a fusion protein comprising AGT (a) and the HaloTag™ (b) is reacted with both objects, each presenting substrates for either AGT or the HaloTag™, respectively. As shown in step (IV.), a covalent crosslink is thereby formed between object (A) and object (B), mediated by the fusion protein of AGT and the HaloTag™. As will be readily understood by the person of routine skill in the art, at least fusion proteins of AGT and ACT, or fusion proteins of ACT and the HaloTag™ are similarly applicable.

FIG. 2. Simultaneous labelling of a HaloTag™-AGT (covalin; 2 μM in PBS) fusion protein with two different fluorophores (simulating objects of interest) and analysis using SDS-PAGE and a laser-based fluorescence scanner (BioRad). Green color represents fluorescence resulting from fluorescein, red color represents fluorescence resulting from Cy3 and yellow color representing fluorescence from fluorescein and Cy3. Lane 1: Labelling of HaloTag™-AGT (covalin; 2 μM in PBS) with fluorescein (green) using commercially available diAcFAM (3 μM, Promega: “HaloTag™ diAcFAM ligand”), the substrate for the HaloTag™. Lane 2: Labelling of HaloTag™-AGT (covalin; 2 μM in PBS) with fluorescein (green) and Cy3 (red) using commercially available diAcFAM (3 μM, Promega), the substrate for the HaloTag™, and BG-Cy3 (3 μM), the substrate of AGT; yellow color demonstrates that the protein is labelled with both fluorophores. Lane 3: Digestion of the sample used for Lane 2 by addition of PreScission™ Protease (0.4 units; GE Healthcare). The protease cleaves HaloTag™-AGT (covalin) at the linker between the proteins, generating fluorescein-labelled HaloTag™ (about 40 kDa, green) and Cy3-labeled AGT (about 20 kDa, red). This control serves to verify the specificity of the labelling.

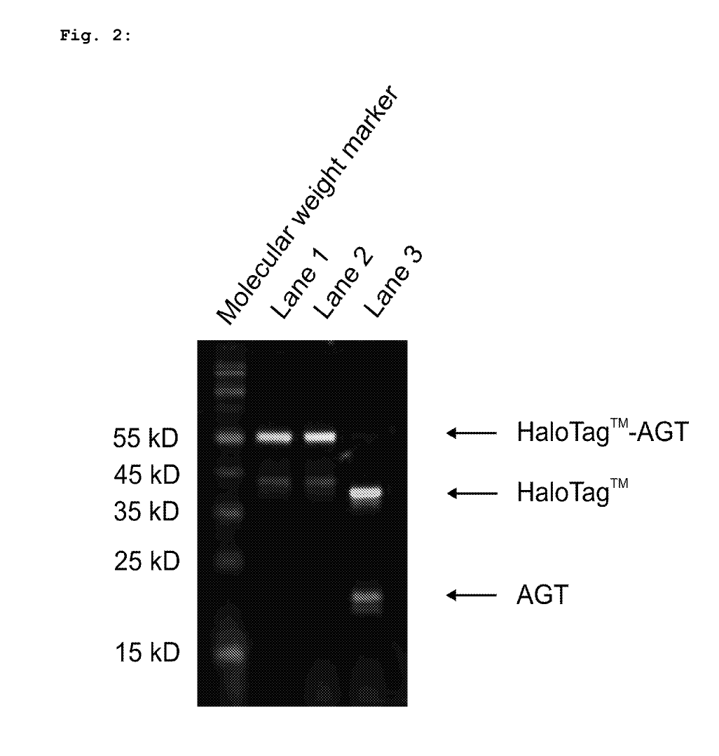

FIG. 3. General scheme for selective bioconjugations through covalin (SEQ ID NO:2). (a) Mechanism of SNAP-tag. (b) Mechanism of HaloTag. (c) Conjugation of two objects displaying either benzylguanine (BG) or primary chloride groups through covalin, a fusion protein of HaloTag and SNAP-tag. (d) SDS-PAGE and analysis through in-gel fluorescence scanning of covalin incubated with: BG-DAF (lane 1), Halo-DAF (lane 2), BG-547 (lane 3), Halo-DAF and BG-547 (lane 4), Halo-DAF, BG-547 and PreScission protease (lane 5). Band quantification of lanes 1 and 2 shows that the ratio of lane 1 over lane 2 is equal to 0.96 (average of two experiments).

FIG. 4. Covalin-dependent labeling of proteins and cell surfaces. (a) 12CA5 derivatized with primary chloride (3 μM 12CA5) was incubated with covalin (10 μM) and BG-547 (15 μM). Aliquots of the reaction mixture drawn at indicated time points were analyzed by SDS-PAGE and in-gel fluorescence scanning. (b) HRP derivatized with BG (3 μM HRP) was incubated with covalin (3 μM) and Halo-DAF (4 μM). Aliquots of the reaction mixture drawn at indicated time points were analyzed by SDS-PAGE and in-gel fluorescence scanning. (c) Detection of different dilutions of recombinant ACP-HA by Western blotting using 12CA5-covalin-HRP (0.17 μg/ml in 12CA5) or a commercially available 12CA5-HRP conjugate (0.15 μg/ml; Roche Molecular Biochemicals). (d) Analysis of a mock pull-down experiment using 12CA5 immobilized on magnetic beads via covalin and incubated with an equimolar mixture of ACP-HA and ACP-Cam-PCP. ACP-HA and ACP-Cam-PCP were both labeled via ACP with Cy3. The ratio of ACP-HA over ACP-Cam-PCP at different stages of the pull-down was determined by SDS-PAGE and subsequent in-gel fluorescence scanning. Lane 1: before incubation of the protein mixture with the derivatized beads; lane 2: after incubation with the derivatized beads; lane 3 and 4: wash fractions; lane 5: elution of captured proteins from the beads. (e-h) Micrographs of derivatized and non-derivatized CHO cells incubated with covalin and a fluorescent covalin substrate. (e) CHO cells derivatized with BG and first incubated with covalin (10 μM) and then with Halo-DAF (2 μM). (f) CHO cells not derivatized with BG and first incubated with covalin (10 μM) and then with Halo-DAF (2 μM). (g) CHO cells derivatized with primary chloride and first incubated with covalin (10 μM) and then with BG-547 (2 μM). (h) CHO cells not derivatized with primary chloride and first incubated with covalin (10 μM) and then with BG-547 (2 μM). Scale bar, 10 μm. Identical microscope settings were used in (e) and (f), and (g) and (h).

### DETAILED DESCRIPTION OF THE INVENTION

As briefly outlined above, the invention provides a fusion protein comprising at least a first protein and a second protein, wherein both the first and the second proteins are, based on their mechanism of enzymatic catalysis, capable of forming a covalent bond with given substrates, and which first and second proteins are of different substrate selectivity, preferably of different substrate specificity; with the provisio that the fusion protein is not ^{M}AGT-DEVD-^{L}AGT or ^{L}AGT-DEVD-^{M}AGT (disclosed in Heinis et al., ACS Chemical Biology 1(9), 2006, 575-84, and further references therein; the disclosure of these documents is incorporated by reference into this application with respect to ^{L}AGT-DEVD-^{M}AGT and ^{L}AGT-DEVD-^{M}AGT).

The term “protein or polypeptide which, based on its structure and function, is capable of forming a covalent bond with a given substrate”, or equivalent wording, is here and henceforth understood as follows: Polypeptides or proteins which possess i) at least one defined substrate binding region for the given substrate, and ii) at least one region, which allows for the irreversible transfer of (part of) the substrate which is bound in the substrate binding region onto an aminoacid residue of the protein. The respective protein or polypeptide possesses a reactivity for its substrate that allows its specific and covalent labelling in the presence of other proteins. Necessary reactivity is here defined as a rate for the covalent bond formation between the polypeptide and the given substrate that is at least 100 times, preferably 1′000 times, most preferably 1′000′000 times faster than the reaction of the reference proteins not possessing such a substrate-binding region such as bovine serum albumin (BSA), chymotrypsin and any of the 20 natural amino acids with this substrate. The respective proteins thus get permanently modified. The respective proteins are, due to their permanent modification, sometimes referred to as “suizymes”. Examples of “suizymes” are ACT and AGT, which transfers an alkyl group from an alkylguanine in an S_{N}2 reaction onto one of its own cysteines, resulting in an irreversibly alkylated protein. Another example is the HaloTag™, which is a genetically engineered hydrolase, in which the release of an intermediate by hydrolysis is impaired:

R—Cl+HaloTag™→R-HaloTag™+Cl⁻—H₂O→ no hydrolysis as in wildtype hydrolase (→R—OH+HaloTag™+H⁺+Cl⁻).

A characteristic of all such “suizymes” is the nucleophilic displacement of a leaving group from the substrate by an amino acid residue of the “suizyme”.

These fusion proteins serve as useful tools in various applications by allowing for selective, preferably highly selective, most preferably specific and covalent crosslinking of two objects. Towards this end, only the desired objects need to be modified, either in vivo or in vitro, with substrate moieties for the respective protein of the fusion protein and be then reacted with the fusion protein, either in vivo or in vitro. The two reactions leading to covalent linkage of the two objects can be carried out stepwise or, even more preferably, simultaneously, even in complex mixtures.

It is to be noted that, dependent on the number of presented substrate moieties on the respective objects, various scenarios can be generated: in a first scenario, a 1:1 ratio of the crosslinked objects can be achieved by each of the objects presenting only one single substrate moiety. In yet a further scenario, ratios of 1:X with X>1 can be easily achieved when one of the objects presents X substrate moieties. Moreover, complex networks can be generated when both the first and the second objects present more than one substrate moiety for the respective protein of the fusion protein. Of course, alternatively or additionally, the fusion protein may be provided with multiple copies of the first or the second protein, or even with yet further different protein(s) of different selectivity or specificity. A possible application for the connection of multiple copies of one object to one copy of the other object is the labelling of an antibody with multiple copies of the protein horseradish peroxidase. This would increase the sensitivity in peroxidase-based ELISA assays.

Concluding, the fusion proteins according to the present invention open up new horizons in various aspects:

First, antibodies can be more easily and flexibly derivatized than with the presently known approaches. The general techniques of antibody handling and derivatization are known in the art; e.g. from (Hermanson, G. T. “Bioconjuation techniques”, Academic Press, San Diego, USA; 1996), incorporated by reference herein. In a straight-forward strategy according to the present invention, the antibody is first e.g. labelled with a substrate for one protein of the fusion protein (in case of AGT, e.g. with a benzylguanine derivative). For example, the antibody is incubated with BG carrying an activated ester group (i.e. N-hydroxysuccinimide esters; commercially available from Covalys Biosciences), leading to formation of stable amide bonds between the BG and lysine side chains and the aminotermini. Subsequently, the antibody only needs to be incubated with the corresponding fusion protein as outlined above, and with a second object which is similarly modified with a substrate moiety for the other protein of the fusion protein, respectively. As will now be evident to the person of routine skill in the art, one single modified antibody and one single fusion protein can be used for a vast variety of applications, depending only on the nature of the second object which is chosen. To mention only some of the possible applications, the second object can be e.g. other proteins, cells, solid surfaces, labels, viruses, quantum dots, any spectroscopic probes useful for imaging technologies in vivo such as MRI or PET, for fluorescence microscopy, radioactive probes, affinity probes such as biotin or digoxigenin, DNA or deoxyribo-oligonucleotides, RNA or ribo-oligonucleotides, antibodies, autofluorescent proteins. The coupling reactions between the fusion protein and their substrates are very fast (approx. 10⁴ sec⁻¹ M⁻¹) and can be performed due to their selectivity or preferably specificity even in complex mixtures, without the need for any purification steps, thus greatly facilitating the handling and increasing the flexibility of usage of a given antibody.

For the modification of cells (here the first object), the cells can be derivatized with a substrate for one of the fusion proteins e.g. by using an activated N-hydroxysuccinimide ester of said substrate using standard procedures such as described in the manual for the biotinylation of cells using an N-hydroxysuccinimide ester of biotin (“Cell surface protein isolation Kit”; Pierce, part of Thermo Fischer Scientific). The modified cells are then simply incubated with the fusion protein and the second object.

According to preferred embodiments, the first and the second proteins are of orthogonal substrate specificity, i.e. the substrate for the first protein of the fusion protein is not at all a substrate for the second protein of the fusion protein, and vice versa (e.g., the combination of an AGT and the HaloTag™); thus, the coupling of both objects to the fusion protein is fully directional, even in complex mixtures and even if carried out simultaneously. Consequently, the formation of homodimers can be reliably prevented. In any case, if the first and the second proteins are not of completely orthogonal substrate specificity, at least a substantially non-overlapping substrate selectivity is required, i.e. both proteins must not exhibit more than 5% reactivity against the substrate of the respective other protein, preferably not more than 3%, most preferably not more than 1%.

In currently preferred embodiments, one of the said proteins of the fusion protein is an O⁶-alkylguanine-DNA alkyltransferase or a genetically engineered, functional derivative thereof, and the other of the said proteins is either
i) an O⁶-alkylguanine-DNA alkyltransferase, or a genetically engineered derivative thereof, which O⁶-alkylguanine-DNA alkyltransferase exhibits, in comparison to the first protein, a different reactivity against at least one O⁶-alkylguanine-DNA substrate (AGTs with altered reactivity are known in the art, cf e.g. WO 2004/031404 (PCT/EP03/10859), WO 2004/031405 (PCT/EP03/10889) and WO 2005/085431 (PCT/EP2005/050889), the disclosure of these documents being incorporated by reference into the present application especially with respect to AGTs with altered reactivity as disclosed therein); or
ii) ACTs, that specifically react with benzylcytosine BC derivatives and which allow the labelling of ACT fusion proteins using BC derivatives the same way as AGT fusion proteins can be labelled with alkylguanine, especially benzylguanine (hereinafter BG) derivatives, cf patent application number EP06117779, entitled “Labelling of fusion proteins with synthetic probes”, incorporated herein by reference. Importantly, the reactivity of ACT versus BG derivatives is below 1% of the reactivity versus BC derivatives and the reactivity of AGT versus BC derivatives is below 1% the reactivity of AGT versus BG. ACT and AGT thus have substantially non-overlapping substrate selectivity.
iii) a genetically engineered derivative of a hydrolase, in which the hydrolysis step is impaired (such as the above-mentioned HaloTag™).

On the DNA level, the fusion protein can be encoded by DNA selected from the group consisting of genomic DNA, cDNA and recombinant DNA.

In currently preferred embodiments, an expression cassette encoding for a fusion protein as outlined above is provided in an expression vector which is known as such in the art. For example, recombinant DNA encoding for the said fusion protein can be incorporated in any suitable expression vector such as e.g. the pET vectors (Novagen) in which the gene of the fusion protein is under the control of the T7 promoter.

Moreover, the invention provides a prokaryotic or eukaryotic host cell line, enabled to functionally express a fusion protein as outlined above and/or transformed with a an expression vector as outlined above. The cell line can thus be e.g. an insect cell line, a yeast cell line, a bacterial cell line or a mammalian cell line. In specific embodiments, the cell line is a S. cerevisiae or an E. coli cell line.

Yet a further aspect of the present invention relates to a kit of parts, comprising:
i) a fusion protein comprising at least a first protein and a second protein, wherein both the first and the second proteins are, based on their structure and function, capable of forming a covalent bond with given substrates, and which first and second proteins are of substantially non-overlapping substrate selectivity, preferably of different substrate specificity; and/or
   an expression vector containing an expression cassette encoding for the said fusion protein; and/or
   a prokaryotic or eukaryotic host cell line enabled to functionally express a fusion protein; and
ii) at least one molecule comprising a substrate moiety for at least either the first or the second protein of the said fusion protein.

By providing the molecule comprising a substrate moiety for at least either the first or the second protein of the said fusion protein (lit. ii), above), the user is enabled to individually use the kit-of-parts for his/her specific purpose by reacting the said molecule with his/her object of interest, thereby transferring a substrate moiety for the fusion protein onto the said object. Preferably, the kit-of-parts comprises both a molecule with a substrate moiety for the first protein of the said fusion protein, and a molecule comprising a substrate moiety for the second protein of the said fusion protein. Typical groups that can be used to couple the substrate to the objects are e.g. activated esters that can react with amino, hydroxyl or thiol groups of the objects; maleimides that can react with thiol groups of the objects; or aldehydes that can react with amino groups of the objects. The person of routine skill in the art will easily choose suitable reactive groups which are able to form a covalent bond under the conditions of the desired application.

In yet further preferred embodiments, the kit-of-parts comprises either a first object already modified with a substrate moiety for the first protein of the said fusion protein; or a second object modified with a substrate moiety for the second protein; or both a first object modified with a substrate moiety for the first protein of the said fusion protein and a second object modified with a substrate moiety for the second protein. Thus, ready-to-use objects may be provided, which proves especially useful for the user e.g. in the case of antibodies, solid surfaces/supports, etc.

Consequently, in yet another aspect of the present invention, a method of crosslinking two objects of interest is provided, comprising the steps of:
i) providing a fusion protein comprising at least a first protein and a second protein, wherein both the first and the second proteins are, based on their structure and function, capable of forming a covalent bond with given substrates, and which first and second proteins are of substantially non-overlapping substrate selectivity, preferably of different substrate specificity;
ii) providing a first object of interest, comprising a substrate moiety for the first protein of the said fusion protein, and providing a second object of interest, comprising a substrate moiety for the second protein of the said fusion protein;
iii) reacting said first protein of the fusion protein with the substrate moiety of said first object, and reacting said second protein of the fusion protein with the substrate moiety of said second object, thereby covalently crosslinking the first object to the second object via the said fusion protein.

The first and the second objects are chosen from the group consisting of spectroscopic probes, affinity handles, receptors, oligonucleotides, solid phases, proteins, enzymes, antibodies, DNA, RNA, carbohydrates, lipids, cells, viruses, quantum dots, carbon nanotubes, radioactive molecules, molecules for magnetic resonance imaging, molecules for positron emission tomography, molecules for fluorescence spectroscopy in vitro and in vivo.

In an especially preferred embodiment of the present invention, the above method of crosslinking two objects of interest is used for derivatization of an antibody, wherein either the first object or the second object is an antibody, and the respective other object is chosen from the group consisting of labels, affinity handles, enzymes, proteins, receptors, oligonucleotides, solid phases, antibodies, cells.

In a further particularly useful embodiment of the present invention, the above method of crosslinking two objects of interest is used for derivatization of a cell, wherein either the first object or the second object is a cell, and the respective other object is chosen from the group consisting of labels, affinity handles, receptors, oligonucleotides, solid phases, antibodies, cells.

An additional aspect of the present invention relates to a method of modifying a first object and a second object, for use with a fusion protein comprising at least a first protein and a second protein, wherein both the first and the second protein are, based on their mechanism of enzymatic catalysis, capable of forming a covalent bond with given substrates, and which first and second proteins are of different substrate selectivity, preferably of different substrate specificity, comprising the steps of:
i) transferring to the first object a substrate moiety for the first protein of the said fusion protein; and
ii) transferring to the second object a substrate moiety for the second protein of the said fusion protein.

Of course, the fusion protein of the first and second protein can be designed by genetic engineering to allow for subsequent cleavage by a protease. Towards this end, a protease site can be introduced e.g. in between the first and the second protein; the fusion protein may thus be specifically cleaved again e.g. after crosslinking of the two objects, if desired so, e.g. for control experiments.

The person of routine skill in the art will readily recognize that yet further peptides or proteins may advantageously be incorporated in between the first and the second protein, in order to impart yet further functionalities. For example, autofluorescent proteins such as green fluorescent protein (GFP) or red fluorescent protein (RFP) might be incorporated in between the first and the second protein, in order to facilitate traceability of the fusion protein. Moreover, proteins could be incorporated in between the first and the second protein that change conformation upon an external stimulus or signal, e.g. calmodulin (upon binding of calcium) or glucose binding protein (upon binding of glucose, respectively), or yet further ligand-binding proteins, in order to enable on-demand conformational changes of the fusion protein, which results in a change of distance between the two objects crosslinked by the fusion protein.

### Preferred Embodiments

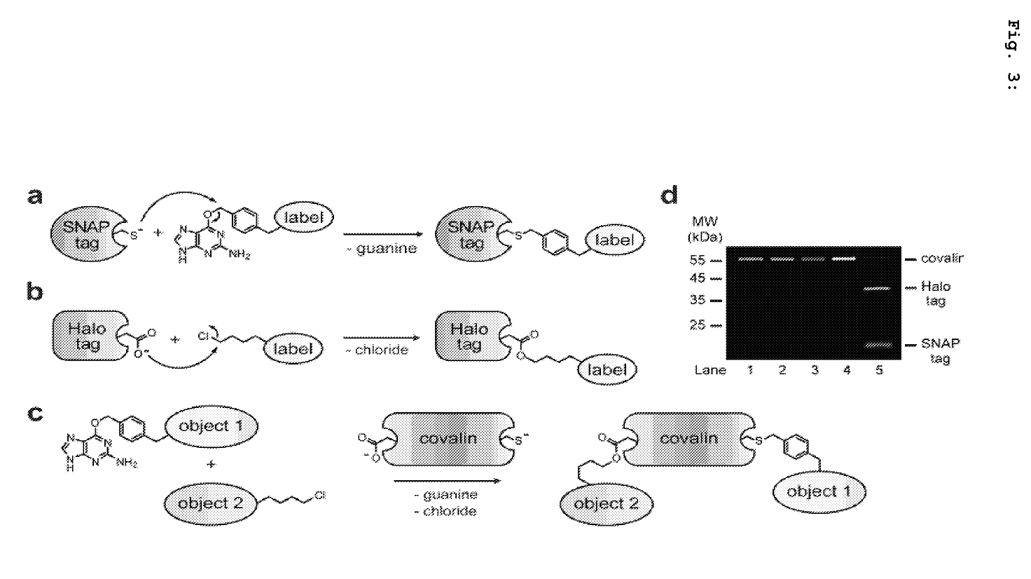

As an adaptor protein for covalent and specific self-assembly of higher structures from different components a fusion protein composed of two self-labeling proteins with non-overlapping substrate specificities was constructed (cf FIG. 3 a-c). The first of the self-labeling proteins is a mutant of human O⁶-alkylguanine-DNA alkyltransferase (abbreviated as SNAP-tag), a monomeric protein of 182 residues that specifically reacts with benzylguanine (BG) derivatives (FIG. 3a); Keppler, A. et al. Nat Biotechnol 21, 86-9 (2003). The second self-labeling protein is a mutant of a bacterial dehalogenase (abbreviated as HaloTag), a monomeric protein of 293 residues that specifically reacts with primary chlorides (FIG. 3b); Los, G. V. & Wood, K. Methods Mol Biol 356, 195-208 (2007). Various substrates and precursors for the straightforward preparation of substrates are commercially available for SNAP-tag and HaloTag and both tags have been used for the labeling of a variety of different fusion proteins in vitro and in living cells. The high selectivity of the two tags for their substrates and the speed of the two labeling reactions, which allows an efficient labeling even at nanomolar concentrations, make SNAP-tag and HaloTag suitable candidates for the creation of an artificial adaptor protein (FIG. 3c). A fusion protein of HaloTag and SNAP-tag with an N-terminal His-tag for purification and a peptide linker containing a PreScission protease (GE Healthcare, formerly Amersham Biosciences) cleavage site was expressed in E. coli. The size of the fusion protein (55 kDa; abbreviated as covalin; cf SEQ ID NO:2) is comparable to that of tetrameric streptavidin (54 kDa). Incubation of covalin with BG-547 and HaloTag diAcFAM ligand (Halo-DAF), substrates for the labeling of SNAP-tag and HaloTag with respectively DY-547 (a fluorophore commercialized by Dyomics, structurally similar to Cy3) and fluorescein, resulted in the labeling of covalin with both fluorophores (FIG. 3d).

BG-547 possesses the following structural formula:

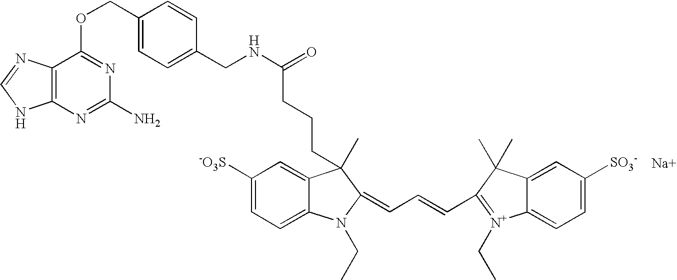

The HaloTag diAcFAM ligand possesses the following structural formula:

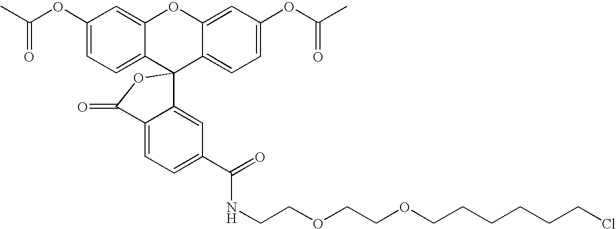

For the reaction mechanisms of both substrates, it is referred to FIG. 3, a-b.

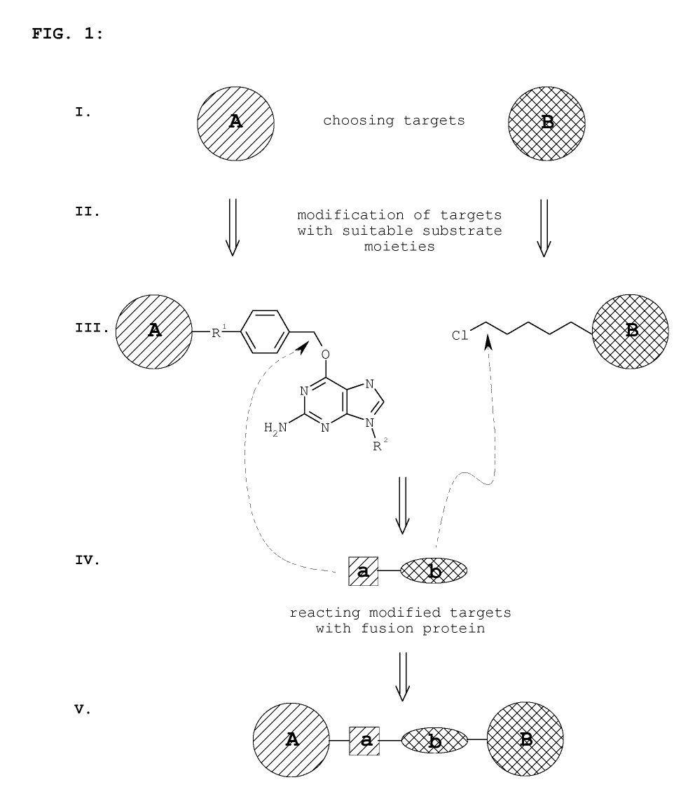

Digestion of the labeled protein with PreScission protease yielded DY-547-labeled SNAP-tag and fluorescein-labeled HaloTag (FIG. 1d), showing that covalin has two independent self-labeling sites. Incubation of covalin with either BG-DAF or Halo-DAF, substrates for the labeling of SNAP-tag and HaloTag with fluorescein, yielded fluorescein-labeled covalins with almost identical fluorescence intensities (±5%; FIG. 3d), indicating that in covalin SNAP-tag and HaloTag are active to the same extent.

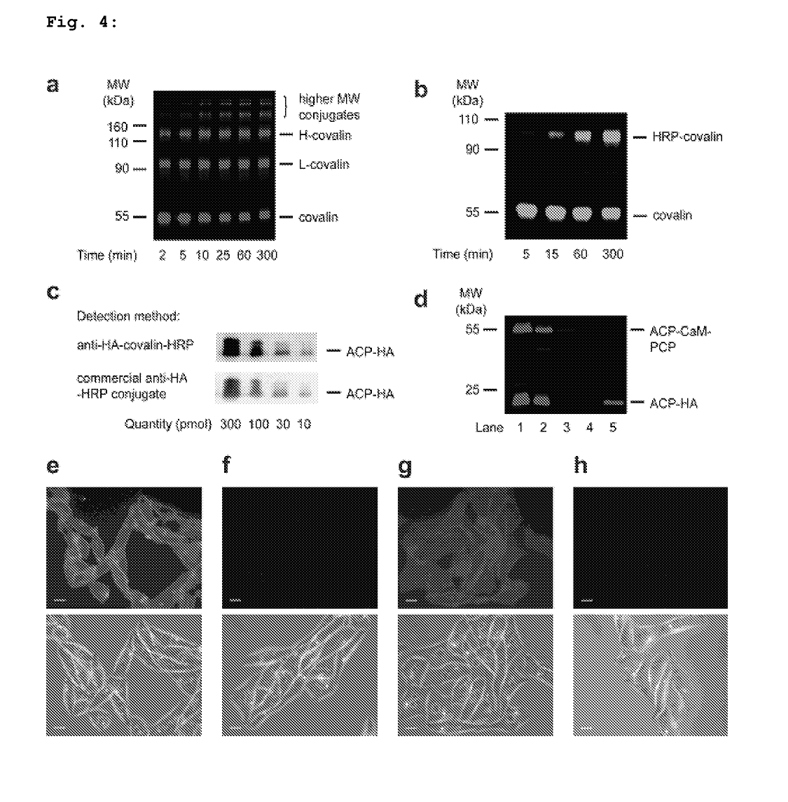

To demonstrate how covalin can be used for covalent self-assembly of higher structures, the conjugation of an antibody to different molecular probes and objects via covalin was attempted. Using covalin for the generation of such conjugates first requires chemical labeling of the antibody with one of the two covalin substrates. Subsequently, the antibody can be functionalized through simple incubation with covalin and the molecular probe or object of choice. Towards this end, the monoclonal anti-HA antibody 12CA5 was labeled with primary chloride through incubation of the antibody with the corresponding, commercially available N-hydroxysuccinimide (NHS) ester, a labeling strategy that should result in antibodies displaying varying amounts of primary chlorides; Hermanson, G. T. Bioconjugation Techniques, (Academic Press, London, UK, 1996). To label 12CA5 with a synthetic fluorophore, the derivatized antibody (3 μM) was incubated with covalin (10 μM) and BG-547 (15 μM). Aliquots of the reaction mixture drawn at different time points were then analyzed by SDS-PAGE and in-gel fluorescence scanning (FIG. 4a). Under these conditions, the fluorescence labeling of 12CA5 with covalin-DY-547 is near completion after an incubation time of one hour and heavy and light chains conjugated to one or multiple covalin-DY-547 could be detected. Approximately 2.5 covalin-DY-547 are bound per anti-HA antibody, as determined by integration of the fluorescence signals. This experiment illustrates how covalin can be used for the straightforward conjugation of synthetic probes to a derivatized protein. Synthetic probes that can be used as covalin substrates comprise fluorophores with emission wavelengths ranging from 440 to 800 nm, including fluorophores with extremely long emission half-lives for time-resolved fluorescence assays (Bazin, H., Trinquet, E. & Mathis, G. J Biotechnol 82, 233-50 (2002)), and oligonucleotides (Jongsma, M. A. & Litjens, R. H. Proteomics 6, 2650-5 (2006); Stein, V., Sielaff, I., Johnsson, K. & Hollfelder, F. Chembiochem 8, 2191-4 (2007)). Moreover, covalin should greatly facilitate the selective conjugation of two different proteins to each other, as it eliminates the challenge to derivatize one of the two proteins with a reactive group that selectively reacts with the other protein. To demonstrate the potential of covalin for such applications, it was attempted to conjugate 12CA5 via covalin to horseradish peroxidase (HRP) and to use the resulting conjugate in Western blotting. Towards this end, HRP was incubated with a BG-NHS ester. To verify that BG-labeled HRP is a substrate of covalin and to determine the degree of labeling of HRP with BG, derivatized HRP (3 μM) was incubated with covalin (3 μM) and Halo-DAF (4 μM). HRP-covalin conjugates were then detected by SDS-PAGE and in-gel fluorescence scanning (FIG. 4b). In these experiments, 40% of HRP was derivatized with one covalin. The derivatization of HRP with NHS esters is known to be inefficient due to the low number of amino groups available (Hermanson, G. T. Bioconjugation Techniques, (Academic Press, London, UK, 1996)) and no attempts were made to improve the labeling of HRP with BG. To conjugate HRP to the anti-HA antibody, derivatized 12CA5 (3 μM) was incubated with covalin (15 μM) and derivatized HRP (30 μM total HRP) for 5 h, after which the solution was directly stored at 4° C. for later use. To evaluate the activity of the self-assembled 12CA5-covalin-HRP, it was compared to a commercially available 12CA5-HRP conjugate (Roche Molecular Biochemicals) optimized for applications in Western blotting. Using recombinant acyl carrier protein with a C-terminal HA tag (ACP-HA) as the antigen, the self-assembled 12CA5-covalin-HRP and the commercially available 12CA5-HRP conjugate showed comparable sensitivity in Western blotting (FIG. 4c). It can thus be concluded that covalin allows the straight-forward and selective coupling of two different proteins to each other.

Covalin should also permit the covalent and selective immobilization of biomolecules or other objects as both SNAP-tag (Kindermann, M., George, N., Johnsson, N. & Johnsson, K. J Am Chem Soc 125, 7810-1 (2003); Sielaff, I. et al. Chembiochem 7, 194-202 (2006)) and HaloTag (Los, G. V. & Wood, K. Methods Mol Biol 356, 195-208 (2007)) have been successfully used in immobilization experiments. To show the utility of covalin in such applications the immobilization of 12CA5 on magnetic beads for pull-down experiments was attempted. Primary-chloride-derivatized 12CA5 (6 μM) was incubated with covalin (9 μM) and magnetic beads displaying BG. For a mock pull-down experiment, the washed derivatized beads were incubated with an equimolar mixture of ACP-HA and of a fusion protein of ACP with calmodulin and peptidyl carrier protein (ACP-CaM-PCP). For detection, ACP-HA and ACP-CaM-PCP were both labeled beforehand via ACP with Cy3. After several washing steps, protein bound to the beads was eluted with SDS sample buffer and samples of different steps of the pull-down analyzed by SDS-PAGE and in-gel fluorescence scanning (FIG. 4d). The enrichment of ACP-HA over ACP-CaM-PCP in the pull-down was 80-fold and no enrichment was observed when in the above procedure derivatized 12CA5 was replaced by original 12CA5. These experiments illustrate how covalin can be used for the immobilization of appropriately derivatized biomolecules.

The lack of reactivity of the covalin substrates towards other (bio)molecules and the absence of natural substrates for SNAP-tag and HaloTag allows the use of covalin as a specific and easy-to-use adaptor protein even in complex mixtures. Such applications include the covalent conjugation of synthetic probes, biomolecules or other objects to the surfaces of cells or viruses. For a further proof-of-principle experiment, covalin was used to conjugate synthetic fluorophores to the surface of CHO cells. In these experiments, CHO cells were first derivatized either with primary chloride or with BG by a brief incubation of the cells with the corresponding NHS ester. Both NHS esters were utilized in order to test covalin in both orientations. The derivatized CHO cells were subsequently incubated first with covalin (10 μM) and then either BG-547 or Halo-DAF (each 2 μM). Labeling of derivatized cells with either DY-547 or fluorescein was detectable by fluorescence microscopy whereas no labeling could be detected when non-derivatized CHO cells were incubated with covalin and either BG-547 or Halo-DAF (FIG. 4e-h). These experiments demonstrate how covalin can be used to conjugate synthetic fluorophores to cell surfaces. Importantly, the synthetic fluorophores could be easily exchanged for biomolecules or other objects, permitting the straightforward assembly of synthetic structures on living cells and viruses.

In conclusion, covalin is a versatile adaptor protein for the self-assembly of higher structures from molecules or objects displaying appropriate functional groups. Conjugations of different objects via covalin are specific, covalent and yield complexes of defined composition. Streptavidin is up to now the most widely used protein component for the formation of higher structures through self-assembly, as it can stably connect biotinylated objects (Astier, Y., Bayley, H. & Howorka, S. Curr Opin Chem Biol 9, 576-84 (2005); Laitinen, O. H., Nordlund, H. R., Hytonen, V. P. & Kulomaa, M. S. Trends Biotechnol 25, 269-77 (2007)). In contrast to covalin, streptavidin has four identical binding sites and its incubation with different biotinylated objects will therefore lead to a mixture of products. Mutants of streptavidin with a reduced number of binding sites have been described (Howarth, M. et al. Nat Methods 3, 267-73 (2006)); however, these mutants also do not allow a specific conjugation of different biotinylated objects. Moreover, the availability of a large variety of different substrates creates immediate and ubiquitous applications for covalin in nanobiotechnology. Finally, the existence of additional self-labeling protein tags with non-overlapping substrate specificities (O′Hare, H. M., Johnsson, K. & Gautier, A. Curr Opin Struct Biol 17, 488-94 (2007); Gautier, A. et al. Chem Biol 15, 128-136 (2008)) allows for the generation of pairs of orthogonal covalins and covalins with different valencies, thereby creating an entire family of new adaptor proteins.

### Further Experimental Details of the Preferred Embodiments

### Expression of Covalin

### SEQ ID NO:2

The sequence encoding covalin (SEQ ID NO:2) was inserted into the vector pET-15b and the resulting plasmid was transformed by electroporation into E. coli strain Rosetta-gami (DE3). A bacterial culture was grown at 37° C. in LB medium to an OD₆₀₀ₙₘ of 1.0 and expression of covalin was induced by the addition of 1 mM isopropyl-β-D-thiogalactopyranoside (IPTG). The bacteria were grown for an additional 21 hours at 16° C. and then were harvested by centrifugation. The bacteria were lysed by sonication and insoluble protein and cell debris were removed by centrifugation. For the purification of covalin, Ni-NTA (Qiagen) was used according to the instructions of the supplier. Eluted protein was further purified by gel filtration on a Superdex 200 column (GE Healthcare Life Sciences) using 20 mM Tris.Cl pH 8.0, 200 mM NaCl, 4 mM DTT. Glycerol was added to a final concentration of 30% (v/v) and the protein was stored at −80° C. The concentration of the protein was determined using a Bradford assay with BSA as a standard.

Labeling of Anti-HA (12CA5) with Primary Chloride:

HaloTag Succinimidyl ester (O4) ligand (NHS—O4-Cl; Promega) was added to a final concentration of 1 mM (from a 100 mM stock solution in anhydrous DMF) to a solution of 7 μM of 12CA5 antibody (Protein expression core facility, EPFL) in PBS pH 7.3 (137 mM NaCl, 2.7 mM KCl, 4.3 mM Na₂HPO₄, 1.4 mM KH₂PO₄). After 30 minutes at 25° C., the excess N-hydroxysuccinimide ester was quenched for 10 minutes by adjusting the reaction mixture to 10 mM Tris.Cl pH 7.4. The labeled antibody was purified from excess Halotag substrate by using a centrifugal filter device (Microcon YM-30, Millipore). Five washing cycles with 50 mM HEPES pH 7.4 were used to concentrate each time the sample from 500 μl to 20 μl. The derivatized antibody was stored at 4° C. until further use. The concentration of the 12CA5 antibody was determined prior to derivatization by UV spectrophotometry using a standard extinction tion coefficient for IgGs of 1.37 ml·mg⁻¹·cm⁻¹. After derivatization and purification, the concentration was set by using an estimated 90% recovery yield.

Labeling of Anti-HA (12CA5) with DY-547 Via Covalin:

A solution of covalin and BG-547 (1.5 equivalents) was prepared and then mixed with primary-chloride-derivatized 12CA5 to a final concentration of 3 μM 12CA5 and of 10 μM covalin in 50 mM HEPES pH 7.4. Kinetics of the reaction was monitored by taking samples of the reaction mixture after 2, 5, 10, 25, 60 and 300 minutes. After drawing the samples from the reaction tube, they were immediately mixed with one volume of 2×SDS sample buffer and heated to 95° C. for 5 minutes. The samples were then analyzed by SDS-PAGE and subsequent in-gel fluorescence scanning (FIG. 4a).

Labeling of Horseradish Peroxidase with BG:

Commercial horseradish peroxidase (HRP type VIA, Sigma) was dialyzed against PBS pH 7.3. BG-GLA-NHS (Covalys Biosciences) was added to a final concentration of 3 mM (from a 100 mM stock solution in anhydrous DMF) to a solution of 100 μM of HRP in PBS pH 7.3. After 90 minutes at 25° C., the excess N-hydroxysuccinimide ester was quenched for 10 minutes by adjusting the reaction mixture to 10 mM Tris.Cl pH 7.4. The labeled HRP was purified from excess BG by using a centrifugal filter device (Microcon YM-30, Millipore). Five washing cycles with 50 mM HEPES pH 7.4 were used to concentrate each time the sample from 500 μl to 20 μl. After elution from the column, the derivatized HRP was stored at 4° C. until further use.

Labeling of Horseradish Peroxidase with Fluorescein Via Covalin:

A solution of covalin and Halo-DAF (1.3 equivalents) was prepared and then mixed with BG-derivatized HRP to a final concentration of 3 μM of HRP and covalin each in 50 mM HEPES pH 7.4, 1 mM PMSF and 2 μg/ml aprotinin. Kinetics of the reaction was monitored by taking samples of the reaction mixture after 5, 15, 60 and 300 minutes. After drawing the samples from the reaction tube, they were immediately mixed with 2×SDS sample buffer and heated to 95° C. for 5 minutes. The samples were then analyzed by SDS-PAGE and subsequent in-gel fluorescence scanning (FIG. 4b).

Procedure for Western blotting:

The antibody-peroxidase conjugate was constructed with primary-chloride-labeled 12CA5 and BG-labeled HRP (both prepared as described above) crosslinked via covalin. The crosslinking reaction was carried out by incubating 3 μM derivatized 12CA5, 15 μM covalin and 30 μM derivatized HRP in 50 mM HEPES pH 7.4 with 1 mM PMSF, 2 μg/ml aprotinin, 1 mM DTT and 0.1% BSA for 5 h at 25° C. The reaction mixture was stored at 4° C. until final use. An HA-tagged recombinant acyl carrier protein (ACP-HA) was serially diluted and loaded in duplicate on a single SDS polyacrylamide gel. The proteins were then transferred to a PVDF membrane (Immobilon-P, Millipore) according to the supplier's instructions. After blocking with skim milk (3% in TBS (20 mM Tris.Cl, 500 mM NaCl) pH 7.5) for 90 minutes, the membrane was cut into two halves each composed of an identical serial dilution of ACP-HA. The first part of the membrane was incubated with 1.5 μl of the reaction mixture described above diluted in 4 ml PBS pH 7.3+0.05% Tween-20. This corresponds to a final concentration of 1.1 nM of 12CA5. The second part of the membrane was incubated with 6 μl of commercially available 12CA5-HRP conjugate (100 ng/μl, Roche) in 4 ml PBS pH 7.3+0.05% Tween-20, corresponding to a concentration of 0.15 μg/ml. Assuming a mono-derivatized antibody-HRP conjugate (MW of 12CA5-HRP: 200 kDa), the final concentration would be 0.8 nM. After 90 minutes at 25° C., the incubation was prolonged 12 hours at 4° C. The membranes were washed four times with PBS pH 7.3+0.1% Tween-20 and then detected using commercial chemiluminescent reagents (Western Lightning Chemiluminescence reagents, PerkinElmer Life Sciences) on a Kodak Image Station 440 (Eastman Kodak).

Pull-Down Experiments:

Magnetic beads displaying BG (SNAP-capture magnetic beads, Covalys Biosciences) were washed twice with immobilization buffer (50 mM HEPES, 100 mM NaCl, 0.1% Tween-20, pH 7.4)+1 mM DTT. In a first step, the beads were blocked with bovine serum albumin (BSA) at a final concentration of 3 μg/μl for 30 minutes at 25° C. on a tube rotator. Then, covalin was added to a final concentration of 9 μM for an additional 90 minutes. Finally, primary chloride derivatized 12CA5 antibody was added to a final concentration of 6 μM. The reaction suspension was incubated at 25° C. on the rotator for an additional 13 hours. The beads were then washed five times with immobilization buffer. Two recombinant proteins both containing acyl carrier protein (ACP-HA and ACP-CaM-PCP) were labeled via ACP with Cy3 according to published procedures (George et al., J. Am. Chem. Soc. 126, 8896-8897 (2004)). A bacterial protein extract was prepared from E. coli JM83 according to standard procedures. The magnetic bead suspension in immobilization buffer was adjusted to a final concentration of 5 μM of both Cy3-ACP-HA and Cy3-ACP-CaM-PCP and to a final concentration of 10 μg/μl of bacterial protein extract. This suspension was incubated at 25° C. for 75 minutes on a tube rotator. The beads were then washed five times with immobilization buffer. Elution of the immobilized proteins was performed by adding 2×SDS loading buffer and heating the bead suspension for minutes at 95° C. In parallel to the experiment described above, two control reactions were performed. Both controls were performed in the same way as described for the experiment above except for the following: in control 1 the primary chloride derivatized antibody was replaced by original 12CA5 antibody, in control 2 covalin was replaced by a buffer load. In both control experiments, no enrichment was observed.

Labeling of CHO Cells:

Chinese hamster ovary cells (CHO-9-neo-C5) were grown in DMEM/F12 (Cambrex) supplemented with 10% fetal bovine serum (Cambrex) and antibiotics (Gibco, Invitrogen) (penicillin 100 U/ml and streptomycin 100 μg/ml final concentrations) in humidified atmosphere at 37° C. under 5% CO₂. Thirty-six hours before the derivatization reaction, cells were seeded on ibiTreat μ-Dishes (Ibidi) to a density of 80,000 cells per dish. Just before derivatization, the cells were washed three times with HBSS buffer (Lonza). The cells were either derivatized with BG by adjusting BG-GLA-NHS to a final concentration of 100 μM in HBSS or derivatized with primary chloride by adjusting NHS—O4-Cl to a final concentration of 500 μM in HBSS. Two control experiments were performed in which the activated ester solutions were replaced by a solvent load. The derivatization step was carried out for 30 minutes at 25° C. The reaction was quenched for 15 minutes by adjusting to 50 mM Tris.Cl pH 7.4. The cells were washed three times with HBSS, then twice with HBSS+0.1% BSA. Covalin was added to each plate to a final concentration of 10 μM in HBSS+0.1% BSA and incubated at 25° C. under gentle rocking for 90 minutes. The cells were washed twice with HBSS, then once with HBSS+0.1% BSA. The BG-derivatized cells (and the corresponding control) were incubated with 2 μM Halo-Fl (Halo-DAF deacetylated in 25 mM K₂CO₃, 50% DMSO) in HBSS/0.1% BSA, whereas the primary-chloride-derivatized cells (and the corresponding control) were incubated with 2 μM BG-547 in HBSS/0.1% BSA. After 15 minutes at 25° C., the cells were washed three times with HBSS. The cells were then imaged in HBSS using a Zeiss Axiovert 200 inverted microscope, equipped with an LD “Plan Neofluar” 63×/0.75 corr Ph2 objective and an AxioCam MR digital camera (Zeiss). Zeiss filter sets 10 (excitation 450-490 nm; emission 515-565 nm) and 43 (excitation 545-625 nm; emission 605-670 nm) were used for fluorescence microscopy. Image analysis was performed with the AxioVision 4.0 software (Zeiss).

What is claimed is:

1-15. (canceled)

16. A fusion protein, comprising: a first protein and a second protein, wherein the first protein is capable of reacting with a first substrate and the second protein is capable of reacting with a second substrate to form a covalent bond such that the first and the second proteins have substantially no overlapping substrate selectivity, and wherein the fusion protein is not ^{M}AGT-DEVD-^{L}AGT or ^{L}AGT-DEVD-^{M}AGT.

17. A fusion protein according to claim 16, wherein the first and second protein have substantially different substrate specificities.

18. A fusion protein according to claim 16, wherein the first and the second proteins have orthogonal substrate specificity.

19. A fusion protein according to claim 16, wherein the first protein is selected from the group consisting of an O⁶-alkylguanine-DNA alkyltransferase, an alkylcytosine transferase, or a genetically engineered derivative thereof, and the second protein is selected from the group consisting of:
i) an 0⁶-alkylguanine-DNA alkyltransferase, an alkylcytosine transferase, or a genetically engineered derivative thereof; and
ii) a genetically engineered derivative of a hydrolase, in which the hydrolysis step is impaired.

20. A prokaryotic or eukaryotic host cell capable of expressing a fusion protein according to claim 16.

21. A prokaryotic or eukaryotic host cell according to claim 20, wherein the host cell is transformed with an expression vector containing DNA sequence encoding the fusion protein.

22. A method of crosslinking two objects of interest, comprising the steps of:
i) providing the fusion protein in claim 16;
ii) providing a first object comprising a substrate for the first protein of the fusion protein, and a second object comprising a substrate for the second protein of the fusion protein;
iii) reacting the first protein with the substrate of the first object, and reacting the second protein with the substrate of the second object, and
(iv) covalently crosslinking the first object to the second object via the said fusion protein.

23. A method according to claim 22, wherein at least one of the first and the second objects are chosen from the group consisting of spectroscopic probes, affinity handles, receptors, oligonucleotides, solid phases, proteins, enzymes, antibodies, DNA, RNA, carbohydrates, lipids, cells, viruses, quantum dots, carbon nanotubes, radioactive molecules, molecules for magnetic resonance imaging, molecules for positron emission tomography, and molecules for fluorescence spectroscopy.

24. A method according to claim 22, wherein the first object is an antibody, and the second object is selected from the group consisting of spectroscopic probes, affinity handles, receptors, oligonucleotides, solid phases, proteins, enzymes, antibodies, DNA, RNA, carbohydrates, lipids, cells, viruses, quantum dots, carbon nanotubes, radioactive molecules, molecules for magnetic resonance imaging, molecules for positron emission tomography, and molecules for fluorescence spectroscopy for derivatizing the antibody,

25. A method according to claim 22, wherein the first object and the second object are proteins.

26. A method according to claim 22, wherein the first object is a cell, and the second object is selected from the group consisting of spectroscopic probes, affinity handles, receptors, oligonucleotides, solid phases, proteins, enzymes, antibodies, DNA, RNA, carbohydrates, lipids, cells, viruses, quantum dots, carbon nanotubes, radioactive molecules, molecules for magnetic resonance imaging, molecules for positron emission tomography, and molecules for fluorescence spectroscopy for derivatizing the cell.

27. A kit, comprising at least one of the following:
(a) a fusion protein according to claim 16;
(b) a first object comprising a substrate for the first protein of the fusion protein, and a second object comprising a substrate for the second protein of the fusion protein;
(c) an expression vector containing a DNA encoding the fusion protein; and
(d) a prokaryotic or eukaryotic host cell line capable of expressing the fusion protein;
the kit further comprising a set of instructions.
